(19) United States
(12) Patent Application Publication
(10) Pub. No.: US 2016/0089049 A1
(43) Pub. Date: Mar. 31, 2016

(54) Long Term Measuring and Recording Device for Electrocardiography

(71) Applicant: Sigknow Biomedical Corporation Limited, New Taipei City (TW)

(72) Inventors: Ming-Wei Hung, New Taipei City (TW); Yi-Yuan Chiu, New Taipei City (TW)

(73) Assignee: Sigknow Biomedical Corporation Limited

(21) Appl. No.: 14/846,267

(22) Filed: Sep. 4, 2015

(30) Foreign Application Priority Data
Sep. 25, 2014 (TW) 103133340

(51) Int. Cl. A61B 5/0432; A61B 5/08; A61B 5/00

(52) U.S. Cl. CPC A61B 5/0432; A61B 5/6832; A61B 5/7203; A61B 5/0816

(57) ABSTRACT

A device for long term sensing and recording electrocardiogram includes a housing body, electrodes, a processing circuit, a water-proof film and at least one flexible attach layer. The housing body includes an upper body and a lower body sealed to form an internal space for enclosing the processing circuit. The middle portion of the water-proof film is sandwiched between the upper body and the lower body, and the outer portion of the water-proof film is attached to a person's skin to form a water-proof region. The flexible attach layer is beneath the water-proof film, and the electrodes penetrate the housing body to electrically connect the processing circuit. The electrodes sense electrocardiogram information, which is processed and recorded by the processing circuit. The device is suitable to wear for a long term without any interference such that the user does not feel uncomfortable even during showering, exercising or sleeping.

### CROSS-REFERENCE TO RELATED APPLICATION

This application claims the priority of Taiwan patent application No. 103133340, filed on Sep. 25, 2014, which is incorporated herewith by reference.

### BACKGROUND OF THE INVENTION

1. Field of the Invention

The present invention generally relates to a device for electrocardiogram, and more specifically to a device for long term sensing and recording electrocardiogram, which is provided with a housing body formed of an upper and lower bodies to accommodate a processing circuit, a water-proof film with a middle portion sandwiched between the upper and lower bodies, an outer portion of the water-proof film covering the skin to form a water-proof region, electrodes penetrating the housing body to electrically connect the processing circuit for measuring and storing electrocardiograph information in the processing circuit.

2. The Prior Arts

In general, electrocardiography (ECG) information is one of the most crucial for asserting physiological status, especially for cardiovascular disease, always vital. Once the disease strikes and the long term record of ECG information is unfortunately lack, the patient often can not be successfully treated, or remains serious sequela after recovery. This leads to not only consumption of a great deal of precious medical resource, but also more pain imposing upon the patient due to the long period of subsequent treatment and rehabilitation. Therefore, the related industries of the medical apparatus have made efforts in developing various smart devices for readily and long term measuring and recording the ECG information.

In the prior arts, the ECG device generally comprises the electrodes for measuring the ECG information, which are often attached to a tape and then adhered to a human body such as chest, hand or leg. Some electrical connection lines are used to connect the display unit or the store unit for displaying or storing the ECG information. For convenience, the ECG device is usually configured to have a specific shape like bracelet, watch, bandage or button for wearing on wrist, arm or leg to be pushed by hand. Alternatively, the traditional ECG device has an outlook of block or box, extending to form the electrodes for attaching the human body.

However, the ECG device in the prior arts has a large size and is heavy such that the user easily feels uncomfortable and the correctness of the measured information is affected. Or daily life is greatly influenced by wearing the ECG device, failing to achieve the aim of long term measuring. Most of the symptom of the cardiovascular disease like fortuitous cardiac arrhythmia happens fortuitously on some special occasion, or at the very beginning. It is difficult for the patient to clearly describe the symptom. As a result, the ECG information recorded for a short period of time may miss some available portion, and is not helpful for clinical diagnosis at all.

Therefore, it is greatly needed to provide a new device for long term sensing and recording electrocardiogram, which features a simple integration, a light and compact structure, and a flat shape. The device of the present invention is convenient to attach the specific position of the skin for a long term, and particularly, the housing body accommodates the processing circuit, and provides the upper and lower bodies to sandwich the middle portion of the water-proof film such that the outer portion of the water-proof film covers the skin to form the water-proof region. The electrodes penetrate the housing body to electrically connect the processing circuit for sensing and storing the electrocardiography information, thereby overcoming the above problems in the prior arts.

### SUMMARY OF THE INVENTION

The primary objective of the present invention is to provide a device for long term sensing and recording electrocardiogram comprising a housing body, electrodes, a processing circuit, a water-proof film and at least one flexible attach layer for being directly attached to a human skin to sense and record the electrocardiography information.

The housing body comprises an upper body and a lower body, which are sealed water-proof and form an internal space. The processing circuit is accommodated in the internal space for insulation and protection so as to prevent invalidation with moisture or water drop seeping into the housing body. The flexible attach layer is configured between the upper and lower bodies to serve as a water-proof gasket, thereby further improving water-proof at the connection region.

The electrodes may comprise a positive electrode and a negative electrode, which penetrate the connection region of the upper and lower bodies, and are electrically connected to the processing circuit. The processing circuit comprises a plurality of electronic elements such as controller, memory unit. For instance, the controller is implemented by a microprocessor and the memory unit is a memory chip or a memory card.

The upper and lower bodies of the housing body sandwich the middle portion of the water-proof film, and the outer portion of the water-proof film outwards extends to completely attach the human skin. As a result, the water-proof film and the bottom of the lower body can attach the human skin to form a water-proof region.

In addition, the flexible attach layer is configured to absorb liquid, and the water-proof film is larger than the flexible attach layer such that the covering area of the flexible attach layer is smaller than that of the water-proof film. Specifically, the flexible attach layer is provided under the water-proof film and covered by the water-proof film so as to directly attach the human skin for absorbing sweat or tissue liquid from the human skin. Thus, the flexible attach layer may provide adhesive strength for being fixed on the human skin.

The electrodes are configured between the water-proof film and the flexible attach layer for sensing an electrical signal of the skin. In particular, the electrodes are preferably attached to the chest to sense an electrocardiography (ECG) signal, which is transferred to the processing circuit as electrocardiography (ECG) information, and then stored in the memory unit. The electrodes may further extends to a hollow region of the flexible attach layer, and is provided at the same horizontal plane as the flexible attach layer for directly contacting the skin and sensing the ECG signal. The processing circuit may comprise an event record button, which extends to the bottom of the upper body for implementing a button function. Specifically, the event record button allows a user to make a specific remark on the electrocardiography information by just pressing the event record button once the user feels uneasy or uncomfortable, like chest tightness, palpitation or dizziness. The remark is helpful for analyzing the electrocardiography information to correctly diagnose the root cause.

Moreover, the electrodes of the present invention can measure the respiration signal such that the respiration information are continuously recorded and stored can when the user wears the device of the present invention. Particularly, all the respiration information is analyzed through software package to recognize respiration waveform, respiration rate, and so on.

With a small volume and a water-proof feature, the device of the present invention is thus suitable to wear for a long term without any interference such that the user does not feel uncomfortable even during showering, exercising or sleeping.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view showing a device for long term sensing and recording electrocardiogram according to the embodiment of the present invention;

FIG. 2 is a side view showing the device of the present invention; and

FIG. 3 is an explosive view showing the device of the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

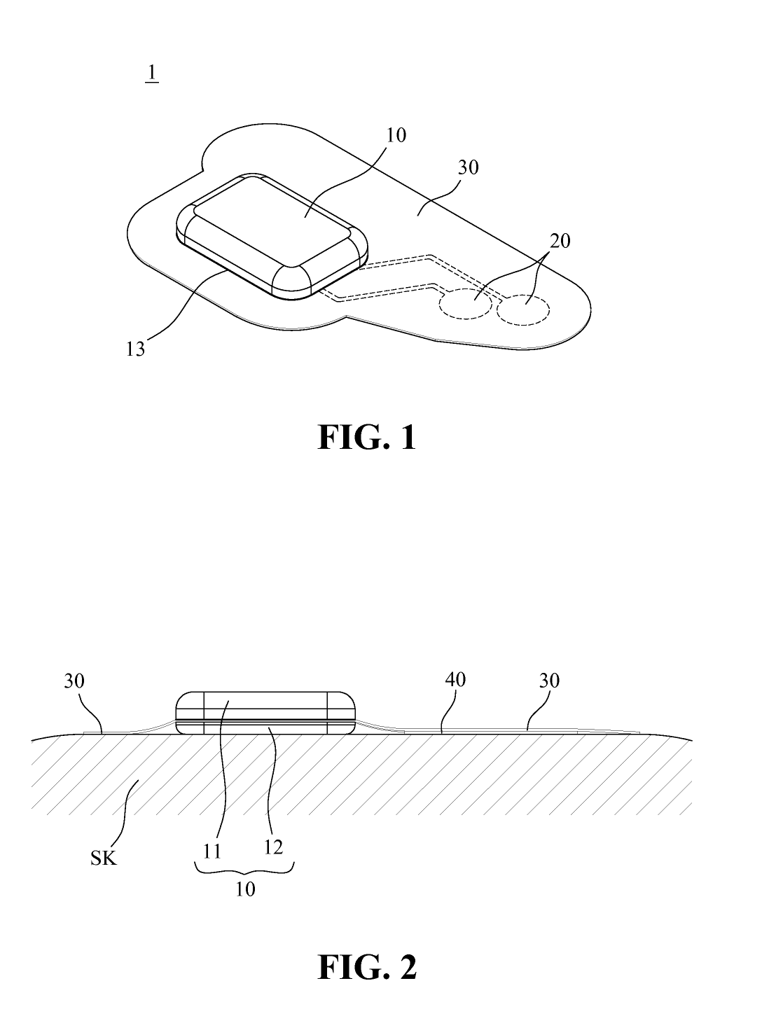

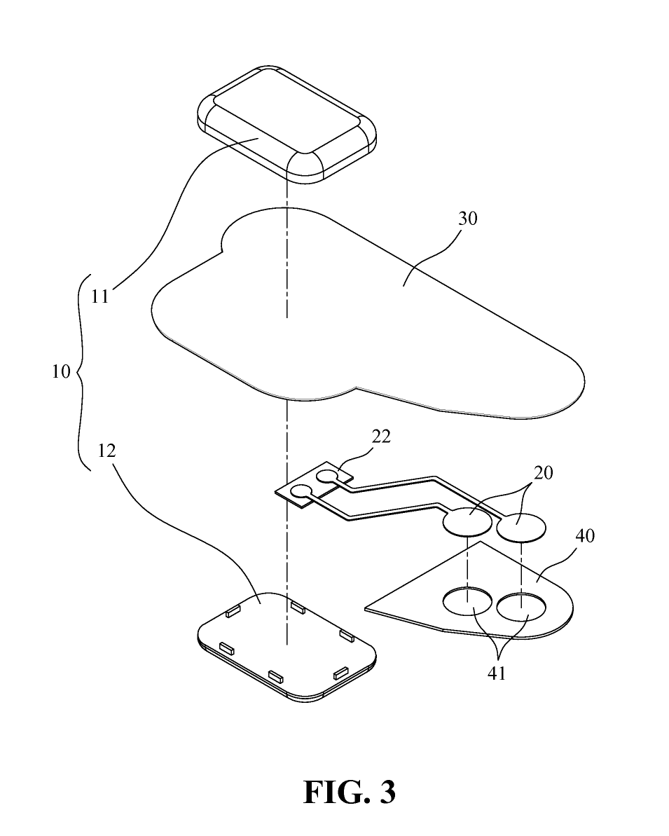

Please refer to FIGS. 1, 2 and 3 illustrating the device for long term sensing and recording electrocardiogram according to the embodiment of the present invention. FIG. 1 shows the perspective view of the device according to the present embodiment, FIG. 2 is a side view and FIG. 3 is an explosive view. As shown in FIGS. 1, 2 and 3, the device 1 for long term sensing and recording electrocardiogram of the present invention generally comprises a housing body 10, at least two electrodes 20, a processing circuit 22, a water-proof film 30 and at least one flexible attach layer 40 for being directly attached to a human skin to sense/measure and record the electrocardiography information. With a smaller size and water-proof, the device of the present invention is suitable for the user to wear for a long term of period without any uneasy or uncomfortable feeling such that it is no need to take off the device even when the user takes a bath, exercises or sleeps.

Specifically, the housing body 10 comprises an upper body 11 and a lower body 12, which are sealed water-proof and form an internal space for accommodating the processing circuit 22 to provide insulation and protection. Thus, the processing circuit 22 is prevented from invalidation without moisture or water drop seeping into the housing body 10. It is preferred that the present invention further comprises a flexible attach layer 13 as a water-proof gasket, which is specifically configured between the upper and lower bodies 11 and 12 so as to further improve water-proof at the connection region of the upper and lower bodies 11 and 12.

The electrodes 20 at least comprise a positive electrode and a negative electrode, which penetrate the connection region of the upper and lower bodies 11 and 12 and are electrically connected to the processing circuit 22. The processing circuit 22 may comprise a plurality of electronic elements such as controller, memory unit. For instance, the controller is implemented by a microprocessor and the memory unit is a memory chip or a memory card.

In addition, the upper and lower bodies 11 and 12 of the housing body 10 sandwich the middle portion of the water-proof film 30, and preferably, the outer portion of the water-proof film 30 at least outwards extends to an extending distance like 2 mm more such that the water-proof film 30 is completely attached to the human skin SK. As a result, the water-proof film 30 and the bottom of the lower body 12 can tightly attach the human skin SK to form a water-proof region.

The flexible attach layer 40 is configured to absorb liquid, like silicon, hydrogel or hydrocolloid dressings, and the water-proof film 30 is larger than the flexible attach layer 40. In other words, the flexible attach layer 40 covers a smaller area than the water-proof film 30. The flexible attach layer 40 is provided under the water-proof film 30 and completely covered by the water-proof film 30. As a result, the flexible attach layer 40 directly attaches the human skin SK for absorbing sweat or tissue liquid from the human skin SK. The flexible attach layer 40 thus provides adhesive strength for being stably fixed on the human skin SK. The electrodes 20 are configured between the water-proof film 30 and the flexible attach layer 40.

More specifically, the electrodes 20 are used for sensing/measuring an electrical signal of the skin SK, and particularly attached to the chest to sense/measure the electrocardiography (ECG) signal, which is then transferred to the processing circuit 22 as electrocardiography (ECG) information. The ECG information can be directly stored in the memory unit of the processing circuit 22, or alternatively, first treated by a data process and then stored in the memory unit. The data process may comprise a digital filter process for filtering noise so as to improve quality of the ECG information.

To further increase sensitivity of the electrodes 20, a conductive adhesive (not shown) is connected to the bottom of the electrodes 20 so as to directly contact the skin SK.

Additionally, the present invention may comprise a reinforcement layer (not shown), which is provided between the electrodes 20 and the flexible attach layer 40 to strengthen stiffness of the electrodes 20. As a result, the reinforcement layer prevents the electrodes 20 from breaking or folded due to excessive stretching or bending.

Moreover, the flexible attach layer 40 specifically comprises a hollow region 41, and the electrodes 20 further extend to the hollow region 41 and are configured at the same horizontal plane as the flexible attach layer 40 such that the electrodes 20 directly contact the human skin SK for accurately sensing/measuring the ECG information.

To reduce friction between the lower body 12 and the human skin SK, a buffer film (not shown) is provided under the lower body 12.

The processing circuit 22 further comprises an event record button (not shown), which extends to a bottom of the upper body 11 for allowing the user to make a specific remark on the electrocardiography information by pressing the event record button when the user feels uneasy or uncomfortable, like chest tightness, palpitation or dizziness. The remark is helpful for subsequent analysis of the electrocardiography information to correctly diagnose the root cause.

In addition, the processing circuit 22 further comprises a primary or secondary battery (not shown) for supplying power for the electronic elements. Thus, the device of the present invention can continuously operate for at least 48 hours. In other words, at least 48 hours of the electrocardiography information is recorded to implement the desired function of long term sensing and recording electrocardiogram.

The device for long term sensing and recording electrocardiogram of the present invention has a flat shape and is applicable to long term wearing like being attached to the left chest of the user to sensing/measuring the electrocardiography information. Also, the device of the present invention is pretty compact is size such that the user does not feel uneasy or uncomfortable while taking a bath, exercising or sleeping. Especially, the outer of the present invention is provided with the water-proof film, which is configured to prevent the device from falling from the skin due to poor adhesive strength by stopping moisture of water drop seeping into the attach region when the user takes a bath. Also, the water-proof film prevents the electrodes from contacting any moisture of water drop so as to maintain signal quality. With the features of compact size, light weight and water-proof, the present invention is considerably suitable for long term wearing, like 7 or even 14 days. Furthermore, the sensed/measured ECG information for the user is instantly recorded and stored in the build-in memory unit while the user wears the device of the present invention. After sensing/measuring, a computer is employed to read out and further analyze the stored ECG information for 7 to 14 days like recognizing ECG waveform, heart rate, and so on, and a final analysis report comes out to assist the doctor to further assert and determine the content of the ECG information for correctness of diagnosis.

Additionally, the electrodes of the present invention can be used to sense/measure the respiration signal through impedance pneumography. The user thus records and stores the respiration information while wearing the device of the present invention, and employs some software to analyze the respiration information like respiration waveform, respiration rate, and so on.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A device for long term sensing and recording electrocardiogram for being directly attached to a human skin, comprising:
a housing body comprising an upper body and a lower body to form an internal space;
a processing circuit accommodated in the internal space of the shell body, comprising a plurality of electronic elements;
at least two electrodes comprising a positive electrode and a negative electrode, extending and penetrating the housing body to electrically connect the processing circuit for sensing the human skin to generate and transfer an electrical signal to the processing circuit as electrocardiography (ECG) information;
a water-proof film having a middle portion sandwiched between the upper body and the lower body, other parts of the water-proof film being provided around the lower body and outwards extending to an extending distance such that at least an outer part of the water-proof film is configured to attach the human skin to form a water-proof space; and
at least one flexible attach layer having a function of absorbing liquid, smaller than the water-proof film,
wherein the electrical signal is directly stored in the processing circuit or first treated by a data process and then stored in the processing circuit, the flexible attach layer is provided under the water-proof film and completely covered by the water-proof film so as to directly attach the human skin for absorbing sweat or tissue liquid from the human skin, the flexible attach layer provides adhesive strength for being fixed on the human skin, and the at least two electrodes are configured between the water-proof film and the flexible attach layer.

2. The device as claimed in claim 1, wherein the electronic element comprises a controller and a memory unit, the controller is implemented by a microprocessor, the memory unit is a memory chip or a memory card for storing the electrocardiography information, the controller is employed to perform the data process, and the data process comprises a digital filter process for filtering noise.

3. The device as claimed in claim 1, wherein the extending distance for the water-proof film is more than 2 mm.

4. The device as claimed in claim 1, wherein the flexible attach layer is formed of silicon, hydrogel, or hydrocolloid dressings.

5. The device as claimed in claim 1, further comprising a conductive adhesive, wherein the electrodes are attached to the human skin through the conductive adhesive.

6. The device as claimed in claim 1, further comprising a reinforcement layer provided between the electrodes and the flexible attach layer to strengthen stiffness of the electrodes.

7. The device as claimed in claim 1, wherein the flexible attach layer comprises a hollow region, and the electrodes further extend to the hollow region and are configured at the same horizontal plane as the flexible attach layer such that the electrodes directly contact the human skin.

8. The device as claimed in claim 1, further comprising a buffer film provided under the lower body to reduce friction between the lower body and the human skin.

9. The device as claimed in claim 1, wherein the processing circuit further comprising an event record button extending to a bottom of the upper body for allowing a user to make a specific remark on the electrocardiography information by pressing the event record button when the user feels uneasy.

10. The device as claimed in claim 1, further comprising a primary or secondary battery for supplying power for the electronic elements.
